(19) United States
(12) Patent Application Publication
(10) Pub. No.: US 2016/0117817 A1
(43) Pub. Date: Apr. 28, 2016

(54) METHOD OF PLANNING, PREPARING, SUPPORTING, MONITORING AND/OR SUBSEQUENTLY CHECKING A SURGICAL INTERVENTION IN THE HUMAN OR ANIMAL BODY, APPARATUS FOR CARRYING OUT SUCH AN INTERVENTION AND USE OF THE APPARATUS

(71) Applicant: Hectec GmbH, Landshut (DE)

(72) Inventor: Jörn Seel, Oberursel (DE)

(73) Assignee: HECTEC GMBH, Landshut (DE)

(21) Appl. No.: 14/921,658

(22) Filed: Oct. 23, 2015

(30) Foreign Application Priority Data
Oct. 24, 2014 (DE) 10 2014 221 738.5
Oct. 22, 2015 (DE) 10 2015 118 050.2
Oct. 22, 2015 (EP) 15191121.1

(51) Int. Cl. G06T 7/00; G06T 19/00; A61B 19/00; G06T 11/00

(52) U.S. Cl. CPC G06T 7/0014; G06T 7/003; G06T 11/008; G06T 19/00; A61B 19/50; A61B 19/5225; G06T 2200/04; G06T 2207/10072; G06T 2207/10116; G06T 2207/30052; G06T 2210/41; A61B 2019/504; A61B 2019/507

(57) ABSTRACT

A method of planning, preparing, supporting, monitoring and/or subsequently checking a surgical intervention in the human or animal body, for introducing at least one implant, in which, with the aid of an imaging assistance means (“IAM”), at least two multidimensional images of at least one portion of the body are displayed, and at least two images which differ with respect to the number of dimensions of the image. The multidimensional images are identified and processed with the aid of the IAM. Information and/or data on implants and/or 2D and/or 3D images of implants is/are supplied to the IAM, which uses an interface to transmit data online during the surgical intervention to equipment for planning, supporting, monitoring and/or subsequently checking a surgical intervention. The IAM is supplied with reference data, target values, standard values and/or general non-patient-specific data and/or information on implants which, before being supplied, is/are compared with standard values.

This application claims priority from German Application DE 10 2014 221 738.5 filed on Oct. 24, 2014, European Application EP 15191121.1 filed on Oct. 22, 2015 and German Application DE 10 2015 118 050.2 filed on Oct. 22, 2015, all of which are incorporated herein by reference.

### BACKGROUND

The invention relates to a method of planning, preparing, monitoring and/or subsequently checking and supporting a surgical intervention in the human or animal body, especially for introducing at least one implant, in which imaging assistance means are employed, using available data, to show multidimensional images of at least a portion of the body, at least two images of which differ with respect to the number of dimensions of the image. The invention relates above all to a possibility of integrating into the method apparatuses for carrying out such an intervention, for monitoring and checking a result or partial result of the planning and the use of the apparatus, and an apparatus for carrying out such an intervention in accordance with the result or partial result of the planning, and the use of the apparatus.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

An implant is a material or part, especially an artificial material or part, which is to be introduced into the body and which remains in the body permanently or at least for a lengthy period of time.

Implants are distinguished according to medical, functional or plastic implants, for example. In contrast to an implant, an exoprosthesis, for example, is attached to the body externally.

Examples of medical implants are cardiac pacemakers, stents and vascular prostheses, artificial hearts or port catheters. In addition, cochlea implants and retina implants are known.

In dentistry, tooth implants with luting anchors replace the natural teeth.

In addition, implants are known which are used to form a depot for a medicinal agent. Once the medicinal agent has been consumed, the implants are replaced.

Another important group is implants which replace damaged joints, for instance. In traumatology, implants are used for the surgical treatment of broken bones. In the case of cranial defects after a trepanation or after a resection, implants made of titanium are fitted as replacement bone for the individual reconstruction of the cranium.

Functional implants are used among other things for monitoring animals or human beings. In this case, RFID chips can be implanted beneath the patient's skin.

Plastic implants are used in plastic surgery for example as replacements for damaged limbs or also to enlarge existing limbs.

Nowadays, most surgical procedures for introducing an implant are planned using a computer-based method for comparing patient data, especially patient images, and possible implants, such as in the form of digital images which can be created and checked interactively using computer model generation software.

Patient images are inter alia X-ray images, which are 2D images and pictures, and volume-based images, such as CTs or MRTs, which are 3D images and pictures.

Volume-based images, i.e. 3D-images and pictures, consist as a rule of several layers of 2D pictures (or 2D tomograms). These can be subdivided into individual 2D tomograms.

Unlike 2D images and pictures, volume-based pictures are already scaled when they are taken. This means that the size of the object imaged and the scale of the image are known. The respective data on the scaling of the picture are stored as a data set together with the volume-based image concerned.

In contrast to this, 2D pictures must as a rule be scaled by the planner/surgeon planning a surgical intervention in the run-up to the planning, in order to establish the scale and possible distortions in the depth of the portion of the patient's body represented.

Subjects of pre-operative planning may, for example, be surgical interventions such as bone resections, the introduction of implants and the selection of their size and type, and various geometrical requirements, including relevant dimensioning, such as height, width, circumference, alignment of certain parts of the skeleton, bones, soft-tissue, other tissue and organs.

In the case of surgical treatment by introducing an implant, 2D patient images, obtained by X-ray imaging as a rule, are preferred to 3D patient images in pre-operative planning, because the patient is subjected to a lower dose of radiation, and a 2D patient image and the associated imaging system is easier to handle. With a 2D patient image, however, there is a degree of uncertainty involved, inter alia in the scaling and in the spatial depth in the viewing direction of the image, i.e. in the projection direction of an X-ray system. One of the effects of this is that it is difficult to establish whether an implant is or would be resting correctly on one of the patient's bones, for example. Further uncertainty relates to whether means for securing the implant, e.g. screws, engage in a bone properly or whether they project above a bone or penetrate or would penetrate into adjacent bones and/or tissue and/or soft-tissue parts.

Planning data in this case may be present in the form of the 2D image data or may be obtained from them by inspecting or measuring the image for example.

In addition to displaying patient images and possible implants, known computer-based systems for pre-operative planning may also offer the planner/surgeon suggestions for selecting a particular implant and the associated instruments to be used in the surgical intervention. The same applies to general types of such implants or instruments.

Methods of this kind are proposed in US 2005 0228 250 A1, WO 2012/113030 A1, US 2012 0 209 394 A1, US 2012 0 027 261 A1 and US 2009 0 299 477 A1, for example.

It is, for example, known to design an individually adapted or patient-specific articulated bearing joint of a knee joint on the basis of the movement profile and the information on soft tissues and ligaments available for a particular patient. In addition, movement information for the patient can be obtained by means of an analysis of the patient's actual gait or by means of computer-model generation software which uses the 3D patient images of the patient's joints and the associated ligaments, muscles and other soft tissues for deriving an analysis of the patient's movements and corresponding recommendations for modifying soft tissue, such as detaching a ligament.

Because of the disadvantages of the 2D patient images described above, however, the evaluation of the operation situation and the adaptation of implants to the individual patient depend to a very great extent on the surgeon's experience and ability to interpret the 2D image data spatially.

Known computer-based systems for pre-operative planning also make it possible, for instance, to view three-dimensional pictures of a patient's anatomy on a computer screen or some other electronic display, to store them on film as a hard copy or to reproduce them on another medium and to view them through direct or indirect illumination or background lighting. A picture may be dimensioned for viewing on any suitable screen size and can be cropped, rotated etc., as the person (e.g. planner/surgeon), viewing the screen wishes.

During the planning process, it is possible to simulate modifying or removing or repairing soft tissue forming part of the anatomy concerned in order, for example, to restore the alignment of a joint or to remove torn or diseased tissue or to cut or repair ligaments or to contemplate natural or artificial ligament transplants. Information about soft tissues may optionally be used as an additional interpretation parameter when selecting a suitable implant.

DE 11 2011 100 810 T5 discloses a method and an associated apparatus for producing an implant. There, a three-dimensional picture of a patient's joint is made, and an implant is selected which is not adapted individually to the patient's joint. The implant is now modified in such a way that it is patient-specific. In the process, at least six angles of the implant are modified. To do so, anatomical and medical information on a patient is integrated with the interactive participation of a surgeon in order to select and make an implant for a particular patient and optionally surgical instruments associated with it, with essentially three options to choose from: all the components of the implant, alignment guides and other disposable instruments may be included in a package which is placed at a surgeon's disposal for a specific patient.

In the known methods of using only 2D patient images or only 3D patient images to plan a surgical intervention, there is, however, the disadvantage that, for example, joint displacements, loads, the absence of loads, or bone and joint deformations, especially pathological deformations of the spine such as kyphosis or lordosis, or different load profiles cannot be displayed in recording systems and therefore cannot be included in the pre-operative planning.

Volume patient images, for example, are only taken when the patient is lying down; in this case, therefore, an image of the patient is frequently obtained when he is not subject to any load.

2D patient images, e.g. X-ray images of the spine, on the other hand, are frequently taken when the patient is standing, because the imaging systems can display that position more easily and can show the part of the body concerned in its actual state, when it is subject to a load.

Especially when interventions on the human or animal spine are being planned, but also on their joints or skeleton parts, it is, however, necessary to be able to take different imaging situations and positions of the patient into account, in particular whether the patient is standing or lying, such as in order to determine the sagittal balance, particularly since the surgical intervention is performed on the patient in a lying position.

It is therefore necessary to make the information obtained from 2D and 3D patient images—especially regarding the presence or absence of loads on skeleton parts, bones, joints, vertebrae, tissues, soft-tissue parts—to the planner of the surgical intervention/the surgeon in a simple manner so that he can preferably view them simultaneously and process them.

The problem of the present invention is to propose an improved method for pre-operative planning, for positioning and/or executing an implant in the human or animal body, with which, in addition to the pre-operative planning, it is possible to monitor the progress of the operation and/or to check the result after the completion of an operation, with which it is possible simultaneously to capture, process and display a plurality of multidimensional patient images or measuring parameters, with which it is possible to pass on the planning data to external devices and equipment and which above all is able to supply data to control an external navigation system. With the improved method it is possible to compare the planning result with the individual steps of the operation to be carried out and the condition of the patient after the operation.

The problem is solved for the method of the invention with the features of the independent claim 1, 2 or 3, for the apparatus used to carry out the method according to the features of claim 38 and for the use of the apparatus in accordance with the invention with the features of claim 42. Dependent claims relate to particular embodiments of the claims listed above.

In the following description, the worked embodiments, the claims and the abstract, the equipment and/or factual circumstances listed below are meant in the following sense:

Imaging assistance means—means equipment which preferably works with electronic means and associated software, which has an interface and makes it possible to receive image data, anatomical data and geometrical data as required from data memories by means of devices or equipment which work with imaging methods and are not connected to the equipment or by means of a manual input, to process those data and to edit them in a software-linked algorithm in such a way that pre-operative planning and/or the output of planning data is/are transmitted to display, auxiliary, measuring or imaging recording equipment used during an operation, or is/are retrieved from them during an operation or is/are retrieved and processed in real time and transmitted back to the equipment connected up.

Imaging method—means any method used before or during an operation, by which information can be obtained about the structure of a human or animal body or parts thereof, or its characteristics, and the pictures recorded by which can be imported, analysed and processed by the imaging assistance means for the purpose of pre-operative planning and/or monitoring during an operation and transmitted via an interface to display, auxiliary, measuring or imaging recording equipment connected to it.

Interface—means equipment which is able, using electric, wireless, optical or magnetic transmission procedures, also including programming algorithms, to transmit data generated by the imaging assistance means to display, auxiliary, measuring, image recording or navigation equipment and/or to receive information from display, auxiliary, measuring or image recording equipment.

Sketch—means an image of the portion of a patient's body which is relevant to the operation, such as is used in general biological or anatomical images. Sketches in this sense are used in particular as aids to enable better visualisation, localisation etc. of the portion of the body displayed, in 2D and/or 3D patient images. Sketches, especially sketches of the spine, can in particular display the portion of the patient's body which is relevant to the operation, especially parts of the skeleton, bones, joints, tissues, soft-tissue parts etc., especially in a lateral image, especially as a 3D model. These sketches inter alia may contain target values, ideal values and standard values of the human or animal anatomy and with the aid of the imaging assistance means compare the values of the individual patient with standard, ideal or target values.

Navigation system—means equipment which is used to assist operations. Preferably it is used to set the geometrical and spatial situation required. It can capture the actual geometrical situation and is able both to receive information from the imaging assistance means via the interface and also to supply information to that means.

Controlling—means retrieving image information generated by the imaging assistance means, going beyond the pre-operative planning process, by means of a manual input, gestures or information from imaging equipment connected up or information from equipment connected up to capture geometrical data.

Patient images are, for example, two or three-dimensional images formed by imaging methods, which can be obtained by known methods. These are preferably 2D images from the use of X-ray procedures or 3D images from the use of CT or MRT. Where the expressions 2D and/or 3D patient images are used below, this is intended to mean not only the actual image itself, but also the underlying data set.

First of all, it is an object of the present invention to unite the information underlying the 2D patient images or 3D patient images in each case or to enable it to be taken into account together in such a way that it is available for visualising and/or processing the other patient image in each case; in particular, it is intended to enable the information in a 3D patient image resulting from the state of the body when it is not under a load to be transferred for visualising and/or processing the 2D patient image, i.e. for altering or modifying in some other way the information and image of the 2D patient image by means of the information and content of the 3D patient image, or vice versa.

It is a further object of the invention to enable scaling of the information in the patient images.

Where not stated otherwise, the terms “information” and “data” are used synonymously in the following.

The advantage of the method of the invention consists in adapting planning steps for a surgical intervention, especially the simulation thereof, to different anatomical modalities of the patient in different images, especially 2D patient images and 3D patient images, by making it possible to display and process a plurality of multidimensional patient images simultaneously. A further advantage is that the method can avoid unfortunate biomechanical results and replace them with an optimised implantation geometry.

According to the invention, the pre-operative situation can be analysed by, among other things, automatically, semi-automatically or manually calculating all the relevant angles and other anatomical or surgical parameters, such as the material properties, size etc. of a possible implant to be introduced and the patient data, and that pre-operative situation can then be used as the basis for the method.

The method of planning, preparing, monitoring and supporting a surgical intervention is carried out on an imaging assistance means, which allows the planner/surgeon to perform archivable, storable and reproducible pre-operative planning and makes his work easier in this way.

The imaging assistance means for the purposes of this invention is capable of visualising, scaling and reading data records available to the planner/surgeon and of displaying at least two multidimensional images of at least one portion of the patient's body, at least two images of which differ at least with regard to the number of dimensions.

A data carrier in this sense may be an integral or external component of the imaging assistance means, such as the memory of a personal computer, notebook, tablet computer, smartphone, memory stick, optical data carrier or cloud system, or other mobile and/or stationary writable storage media.

In accordance with the invention, the planner/surgeon using the imaging assistance means can supply, retrieve, import or download and display simultaneously or in succession a plurality of selectable multidimensional patient images, especially in the form of 2D patient images and 3D patient images.

The imaging assistance means recognises the patient images made available, inter alia with respect to their format e.g. as 3D pictures and/or 3D image data or as 2D pictures and/or 2D image data.

The patient images and/or other patient information may be contained in a PACS system (picture archiving and communication system) or another data memory, for example. They may be stored in electronic and/or digital form, such as on a magnetic storage medium, an optical storage medium or on a semiconductor memory, a so-called cloud system. They may also be stored on a mobile or stationary storage device.

The patient images and patient information may, for example, have been obtained at different times, they may comprise 2D and/or 3D picture data sets of the patient and/or further data, especially on the patient's height, weight and age, and data from earlier interventions, data from earlier examinations and/or data created directly in connection with an operation.

The information may, for example, be supplied to the imaging assistance means electronically, via the internet, using suitable transmission protocols and/or in other ways, especially for retrieval, importing or downloading and displaying.

Data transmission may be effected directly from the hard disk of a computer or by means of some other mobile or stationary data carrier of a hard disk, CD, DVD, flash storage device (e.g. memory stick, compact flash, secure digital card) or a cloud system.

In addition, encrypted or unencrypted transmission is possible by e-mail or other digital transfers to any suitable type of computer device, smartphone, PDA, tablet computer or other devices via which electronic information can be transferred.

It is also conceivable to supply the imaging assistance means with 2D tomograms obtained from a 3D patient image or external 2D tomograms already available from 3D patient images and to provide them for the further processing and/or planning of the surgical intervention and the use of an orthopaedic implant. The same applies, mutatis mutandis, to 3D pictures obtained from 2D patient images.

The imaging assistance means can also derive and calculate 2D tomograms from a 3D patient image and vice versa. In order to display the 2D tomogram, a plane is placed in the three-dimensional body, such as bones, tissues, joints or soft-tissue parts, especially the vertebrae of the spine, which may run horizontally or vertically or at an angle to the axis of the spine. The coordinates of the plane determine the coordinates of the 2D tomogram and make it possible to display the perspective of a particular two-dimensional layer of the three-dimensional body. The 2D data can be calculated automatically.

In addition, the 2D and 3D patient images, which are available to the method, can be displayed in a high-resolution MIP or MPR image with the aid of the imaging assistance means.

Furthermore, in order to carry out the method of the invention, the imaging assistance means can be supplied with further information, such as patient images, dimensioning data, functional data and/or other reference data, target values, standard values and/or general non-patient-specific data and/or information on implants, such as 2D and 3D models of implants, manufacturers' information, article numbers, dimensioning, material, instructions for use etc. of the implants, and/or sketches as well as standard, ideal or target values of the human or animal anatomy.

The above-mentioned data and information may, for example, be stored on a personal computer, notebook, tablet computer, smartphone, data stick, CD or DVD, or in a cloud system, data archive or some other mobile and/or stationary processable storage medium and may be supplied to the imaging assistance means in the way described above.

Patient-specific and general information can be shown, displayed and/or stored especially with the plurality of multidimensional patient images.

In addition, it is possible in accordance with the invention to use the imaging assistance means to process 2D patient images before, during or after they have been supplied, especially to scale them. In the process, dimensions of the portion of the patient's body displayed, especially the spine, and/or the scale of the image are determined automatically, semi-automatically or manually with the aid of the imaging assistance means, such as by using a known reference object, such as a sphere made of non-radiolucent material.

The scaling procedure can be carried out and repeated separately for each individual 2D patient image imported.

The imaging assistance means also enables the planner/surgeon to segment or scale the 3D patient images supplied in different views, sections and/or perspectives. This makes it possible to recognize the volume and not only individual points of the patient images. Segmenting can also take place and be stored in a network or in a cloud system.

Segmenting makes it possible to identify and provide the medical/anatomical name and/or numbering of individual bones, vertebrae, joints, soft-tissue parts, tissues etc. Segmenting also makes it possible to identify and provide the medical/anatomical name and/or numbering of especially the spinal vertebrae, by means of reference models, and target values. It enables the comparison with medical and anatomical ideal and standard values of the human or animal anatomy in order to allow the most ideal planning of the patient anatomy aimed at through the operation, which can be supplied to the imaging assistance means as described above. In addition, with the aid of the imaging assistance means, individual bones, soft-tissue parts and tissue parts in the images can be superimposed or faded out or highlighted.

Segmenting with the aid of the imaging assistance means, i.e. especially identifying and designating individual bones and/or skeleton parts, vertebrae, joints, soft-tissue parts, tissues etc. may in particular be performed automatically and checked manually for example.

Especially in the case of vertebrae, this can be displayed, clarified and/or checked by means of sketches supplied, especially vertebral sketches.

According to the invention, the imaging assistance means can be used to display simultaneously, on one screen, windows with different images, such as different patient images, sketches of the portion of the body which is relevant to the operation, e.g. a spine or a vertebra.

The imaging assistance means controls the structure of the screen in such a way that the screen either has a single window or can be subdivided into more than one window. The screen may be subdivided horizontally, vertically, diagonally or in any other way, especially simultaneously in at least two windows of the same or different sizes.

One image in each case may be displayed in the at least one window. The images may in particular comprise patient images, sketches, especially sketches of the spine, diagnosis monitors and other illustrations or data, such as on implants. Furthermore, patient images of a larger portion of the body, but also individual bones, joints, soft-tissue parts and/or tissues, especially individual vertebrae, may be displayed in a window.

The images of at least two windows can be displayed simultaneously.

The images of at least two windows may in particular differ with respect to the number of dimensions of the images.

In the method of the invention, at least two multidimensional images of at least one portion of the patient's body can be displayed simultaneously in an imaging assistance means, especially in at least one window, and data relevant to the operation can be collected and combined.

These data relevant to the operation may comprise 2D and/or 3D picture-data sets of the patient and/or further data, especially concerning the patient's height, weight, age, data from earlier interventions, and data from earlier examinations and/or data created directly in connection with an operation.

One aspect of the invention consists in the fact that, based on the method of the invention, different 3D patient images and/or 2D patient images can be displayed simultaneously in windows, in particular at least one image of a 2D patient image and at least one image of a 3D patient image in at least two windows of the screen.

In this context, it is possible, with a further aspect of the invention, to use the imaging assistance means to create a spatial coordinate reference between 2D patient images and 3D patient images. In particular, a correlation can be created between the preferably spatial information of data and/or information in 2D patient images and 3D patient images and vice versa. In this way, information from one image can be transferred into another image, displayed and processed.

In addition, the method of the invention further contemplates the possibility of enabling patient images displayed in at least one window to be processed by the planner/surgeon with the aid of the imaging assistance means.

In particular, the planner/surgeon can, for example, activate one of the images in at least one window for processing in a working representation, by clicking with a cursor for instance.

A cursor in this sense is any display, inputting, command initiating and/or processing device. It may, for example, be a mouse, a trackball, a keyboard, the display of a touchscreen or a graphic tablet, especially with a pointer.

At least one of the multidimensional images in this case, in particular at least one 2D image or one 3D image, can be activated with the aid of the imaging assistance means as a working representation, it being possible to create a spatial coordinate reference between the individual images.

In particular, it is possible for the coordinate reference to be transferred automatically from a 2D image to a 3D image and vice versa. This is done in particular independently of the format of the patient image (especially the 3D or 2D patient image), the viewing angle from which the respective image is displayed, or which axial, sagittal, coronal or other section of the patient image the working representation and/or further window show(s).

It is in particular possible, to display a 3D patient image in one window and a 2D tomogram of that 3D patient image in the other window, where the 2D tomogram in particular constitutes an axial section through the spine or the vertebra, for example.

In addition to a 3D patient image, it is, for example, possible to display three 2D tomograms of that 3D patient image, one 2D tomogram of which shows an axial section, the other 2D tomogram shows a sagittal one and the third 2D tomogram shows a coronal section through the respective region of the spine.

Apart from that, it is, for example, also possible for a sketch, e.g. a sketch of the spine, to be shown in one window. During the display at least one further window is shown in an enlarged and considerably more detailed form. In addition, a corresponding excerpt from a patient image with a region of that sketch marked by the planner/surgeon with a cursor can be shown. Apart from that, the patient image itself can be shown.

The marked region may in particular comprise one or more vertebrae of the spine with and without intervertebral disks, it being possible in the sketch to display the marked elements, such as vertebrae and/or intervertebral disks, in different colours.

In the working representation, portions of a patient's body relevant to the operation from different multidimensional patient images may be displayed, measured and/or processed in order to carry out the pre-operative planning.

A cursor can be used to display and/or process patient images, hence to select a working representation in the individual images.

The working representation concerned is used in particular for the processing and/or simulation of a surgical intervention by the planner/surgeon.

Particular regions of patient images, such as bones, joints, soft-tissue parts and/or tissues, can be displayed, measured and/or processed especially with computer assistance, for example with the aid of a cursor or with other pointer means, with the aid of the at least one working representation of the imaging assistance means in correlation with a sketch. In particular, individual vertebrae can be selected and marked. The vertebra selected can be highlighted in colour relative to the other vertebrae.

In a working representation, the planner's/surgeon's processing and/or simulation steps can in particular be performed with computer assistance, such as with the aid of a cursor, or other input methods.

By marking, such as clicking on, a bone, joint, soft-tissue part or tissue in the working representation, especially one or more vertebrae of the spine, the planner/surgeon can in particular carry out planning steps on a region of the spine in a 2D patient image and/or a 3D patient image and simulate individual steps of the operation in the at least one working representation.

Because of the coordinate reference between the 2D patient images and the 3D patient images in accordance with the invention, the planner's/surgeon's various processing steps can be imported into the other images on the basis of the coordinates. Hereby all the processing steps can be displayed, planned and checked in all the images selected, especially for all the positions of the portion of the body concerned, whether under load or without any load and in all the dimensions selected and in comparison with target values and ideal values from sketches for instance.

In addition, for pre-operative planning, especially for simulating a surgical intervention, it is possible to arrange at least one movable model of an implant, which is movable relative to the portion of the body, in one or more of the images of the portion of the patient's body on which the intervention is being performed, which is/are configured as a working representation or representations.

One major aspect of the method of the invention consists in the fact that the work and planning steps which the planner/surgeon carries out in the working representation, especially in connection with a possible implant that has been found—such as positioning the potential implant by means of an implant model, especially on a vertebra of the spine, altering the shape and/or size of the implant model, rotating or displacing the implant model, altering the set angle of an implant model relative to a vertebra, for example, and/or dimensions or measurement results, can be displayed simultaneously and in the corresponding spatial coordinates in each case, preferably by creating a coordinate reference to at least one or more or all the other windows.

The imaging assistance means in particular makes it possible to obtain an all-round view of the portion of the patient's body relevant to the operation by means of the patient images and possible implant models in the respective working representation. In particular, it is possible for images of regions of the body and implant models to be superimposed and faded out, focused, introduced or dimensioned by the planner/surgeon with the aid of the cursor, for example.

The imaging assistance means can be used to evaluate the data supplied, especially the dimensioning, measurement results etc. in the context of a comparison of the individual patient data with sketches or target, ideal or standard values and to check them for deviations between target and actual values in order to achieve an optimal post-operative result for this patient.

With the aid of the imaging assistance means, in particular with the aid of the at least one working representation, it is possible to display specific dimensioning used in the field, such as the length, depth, circumference, height and width and/or density of, for example, a bone and/or part of a skeleton, a tissue, soft-tissue part or organ, in the form of reference points, by means of which reference points on implant models which may be stored in a database can be compared and in this way automatically or manually to select and propose for the patient concerned matching implants, such as cages (implant cages), components, artificial joints, screws, plates, distraction-compression apparatuses, implant components, alignment guides, disposable instruments, securing means, acetabula and shanks for hip implants etc.

In a preferably automatic selection process for planning a surgical intervention, a comparison is made between known and/or identified reference points between implant information and the patient images and is used to determine and propose at least one most suitable implant, which is automatically superimposed in one of the images, e.g. in the 3D patient image, preferably in the at least one working representation, with the aid of the imaging assistance means.

For the purpose of making a precise selection and/or fit of a possible implant, the inner corticalis of the patient, for example, is automatically identified on the patient images with the aid of the imaging assistance means in accordance with the invention. In this case, the imaging assistance means can be used to display, by means of visual, especially coloured, marks, whether measurement results are inside or outside a predetermined standard range.

In addition, the planner/surgeon can make manual changes according to his own criteria for his decision when selecting the implant.

In particular it is possible for the planner/surgeon planning and/or simulating a surgical intervention on the spine to remove at least one part in a patient image, such as a vertebra, by highlighting it, especially by clicking on it with the cursor in the at least one working representation. Furthermore, it is possible to arrange other parts on the vertebra or, for example, to fix an implant on the vertebra by highlighting it, especially by clicking on it with a cursor. Depending on the perspective, the planner/surgeon can rotate, pan and/or tilt a 3D image in a working representation with the aid of a cursor for instance.

It is in particular also conceivable to simulate sections through any body part displayed, such as through vertebrae, and further potential steps of the operation to be planned and carried out in order to establish what effect a particular arrangement of an implant has relative to the internal region of the body part.

A 2D image corresponding to the 3D image can likewise be processed by means of the cursor. In this case, it is possible to scroll through 2D images and/or 2D tomograms e.g. with the aid of the scroll wheel of a mouse or a similar computer-linked pointer mechanism. The image generated in this way is in screen coordinates, i.e. in two-dimensional feature points. As a result, the method of the invention enables a precise orientation and transfer of coordinates both in 2D images and in corresponding 3D images of a portion of the patient's body displayed. The cursor makes it clear which region of a 3D image and/or a 2D image the planner/surgeon is marking and/or planning and/or processing at the time concerned.

Processing steps of this kind, such as in 3D patient images, can preferably be reproduced automatically in their effects, also in 2D patient images, preferably producing a coordinate reference. The same applies, vice versa, when the processing steps are performed on 2D patient images.

According to the invention, it is in particular possible, during the pre-operative planning of operations on the spine for example, to measure the sagittal balance with computer assistance on the basis of the patient images. For this purpose, the planner/surgeon marks reference points in least one of the images in the windows with the aid of the cursor. These are in particular the ventral end point of the base of the sacrum, the ventral-cranial end point of the base of the sacrum and/or the two femur head centre points. Furthermore, the measurement results can with computer assistance be subjected to a target/actual comparison with target, ideal and standard values of the human or animal anatomy and be listed and evaluated in a result list. In this way, it is in particular possible to select and position a potential implant to be introduced according to certain parameters.

For further processing in the position concerned in a 3D or 2D patient image, the planner/surgeon introduces an implant model into the 3D or 2D image with the aid of the cursor in the at least one working representation.

Later on, the implant may, for example, be a screw in a vertebral body of the spine.

A large number of possible parameters are available for the selection of the screw. Selection criteria are, for example, the desired manufacturer of the screw, the type of screw, the screw configuration, the screw material and its size and length.

With the method of the invention, it is first of all possible to define the desired parameters of the screw with the aid of the imaging assistance means. In a next step, the bone relevant to the operation is selected, especially a vertebral body, e.g. in a sketch and/or in a patient image in one of the images in the windows. Selected excerpts can be displayed in at least one image in a further window.

The implant model, such as a screw, which is selected on the basis of predefined parameters can be provisionally placed in a multidimensional patient image in the working representation with computer assistance. Simultaneously, the implant model in the form selected is displayed in further images in the windows at the corresponding spatial coordinates of the patient's body reproduced in the respective images. The positioning of the screw on a vertebra in the other windows corresponds to the arrangement and orientation of the screw on a vertebra in the working representation.

With the aid of the cursor, such as by dragging with the cursor on or along the X, Y and/or Z axis, the positioning of an implant model, such as a screw, can be shifted in the working representation without altering the inclination, direction or angle, and/or it can be rotated on or along circles, which show the axis of rotation or the possible direction of rotation in each case.

The possibility also exists of simulating a hole drilled in a bone, e.g. a vertebra, for an implant model, such as a screw, in the working representation, and the planner/surgeon can lengthen or shorten the depth of the hole.

It is also possible, in the working representation, to use the cursor to simulate the change in the angle of inclination, the angle of rotation and the swivelling angle of the implant model, e.g. a screw, in a bone, such as a vertebra. Hereby the simulation is transferred to other, adjacent bones, especially vertebrae, retaining the simulated shape and quality and retaining the simulated orientation with respect to the bone represented. Preferably the simulation is carried out based on vertebrae of the spine in comparison with the surrounding bones and soft-tissue parts, for example the vertebrae of the spine.

In the way described, different implants and their spatial arrangement can be simulated in the patient image and planned with regard to their effect on and compatibility with adjacent bones, tissues, soft-tissue parts, organs, etc. The surgeon thus has the possibility of planning and/or simulating the optimum implant and the optimum orientation of the implant for each patient.

Another reason why the invention is advantageous is that the results of the surgical intervention, in particular the introduction of at least one orthopaedic implant, can be checked and if necessary corrected by means of the simultaneous display in at least two windows and with the help of the display from different angles or perspectives in further windows. This is done irrespective of whether the simulated action was performed in a 3D or 2D working representation or whether at least one further window is open in addition to the working representation.

A further advantage of the method of the invention is that despite the large number of measurements that can be performed when planning the introduction of the implant, fully automatic recognition of the bones, tissues, soft-tissue parts, joints, vertebrae etc. which are displayed in the patient images is possible, and they can be named.

The method in particular makes it possible to plan operations on the spine, taking into account the Nash-Moe vertebral rotation, the interpedicular distance, the spinal canal width index, lordosis, kyphosis, atlantodental intervals, the diameter of the spinal canal, the vertebral lines, the McGregor line, spondylolisthesis, the intervertebral disk height, the intervertebral disk angle, the instability according to van Akkerveeken, the sacral angle, functional analyses, stability criteria in spondiloretrolysthesis, the arrangement of superior and inferior endplates, the orientation of a plumb line to the centre of gravity of a vertebral body, the cervical centroid line, von Rissergrad, scoliosis according to Cobb or Ferguson, pelvic tilt PT and/or pelvic incidence PI, pelvic angulation PA, pelvic lordosis angle, pelisacral angle PSA and/or sacral slope, C7 plumb line, pelvic thickness CS, pelvic thickness SPT, etc., with which individual steps of a surgical intervention on the spine can be simulated in a working representation. Especially from the anterior/posterior and sagittal views of the spine, it is also possible to determine the apex angle, for example, and the actual angle, and to base the method on them.

The method can inter alia also be used when planning hip replacements. The method makes it easier for the surgeon to select and fit the suitable combinations of acetabulum and shank. In addition, the method of the invention can be used to correct the adduction and/or abduction of the limbs relative to the hip. Furthermore, it is possible to determine and represent the leg length equalisation pre-operatively and post-operatively.

Furthermore, the method of the invention can be used to produce a biometrical analysis and a suggestion for the optimised location of joint centre points. The height, weight and the biomechanical initial analysis of the patient can be taken into consideration automatically. When a biometrical analysis is performed, the method of the invention can be used for example to simulate the pivot point for optimised joint geometry, taking the load situation of a healthy joint as the basis. Dimensioning and measurement results of this kind can be determined and displayed with computer assistance.

When a surgical intervention in a child's hip is being planned (coxometry), the surgical intervention can be based on clinically relevant dimensioning for the assessment of hip joints. In order to take account of differences in national criteria when planning a surgical intervention on a child's hip, the method is based on graduation tables which take these nationally different criteria into consideration, so that the method can be used internationally.

In addition, the method of the invention can also be used for knee endoprostheses. In particular axial misalignments of a knee prosthesis can already be determined and corrected during the pre-operative planning.

Employing the method, it is possible to make an automatic and/or manual correction to the misalignment, of the leg for example, depending on the objective concerned. For this purpose, the mechanical axes that can be expected after the operation, the axis line and all the relevant angles are determined.

According to the invention, the method can also be used when planning an osteotomy.

For this purpose, the targeted transection of one or more bones, e.g. in order to correct misalignments of the leg axes or a hip misalignment, can be planned. The method of the invention also makes it possible to decide on one or more osteotomies in terms of their nature, number, size and location. Femoral or tibial repositioning osteotomies in particular can be planned with the aid of the method, such as single or multiple osteotomies according to the open-wedge method or according to the closed-wedge method.

After a correction has been made, all the joint-related, mechanical load axes and joint tangents can be subjected in accordance with the invention to a target/actual comparison of the patient data with target, ideal and standard values or sketches of the human or animal anatomy in order to achieve the best possible post-operative result for the patient.

In order to plan the osteotomy, the imaging assistance means can join partial images from individual images and/or patient images together into a single image, for example in order to simulate the joining of bone parts. Furthermore, the surgeon can carry out simple image processing steps with the aid of the imaging assistance means for a reconstruction of skeleton elements.

A further advantage of the invention is that when planning the osteotomy, bone segments can be revealed and shifted, and any osteosynthesis implants needed can be placed in position. Misalignment angles, for example, can be calculated according to Dror Paley.

In addition, components suitable and/or necessary for osteosynthesis, such as nails, plates, screws etc., can be selected.

The method can also be used to plan a surgical intervention on shoulders, elbows, hands and fingers, and to prepare the selection and position of implants in the optimum way.

The method of the invention can also enable an improvement in the introduction of forces and the restoration of the physiological muscle play by means of standard ranges determined biomechanically, according to which the implants are anchored.

The same also applies to planning a surgical intervention on the human and/or animal foot, ankle joint or toe, such as when the big toe is misaligned in the base joint and points outwards (hallux valgus).

The method of the invention also enables the planner/surgeon to determine axial misalignments and to correct them automatically or manually.

Measurement results before and after an operation can be displayed according to the method in the at least one working representation.

According to the invention, in addition to the individual working steps also comments and remarks regarding planning can be stored and supplied via the imaging assistance means during the planning process. Apart from that, individual working steps, comments or displays in the working representation can be superimposed and faded out.

According to the invention, the final planning result of a possible surgical intervention to be planned and/or planning steps for the planning process, i.e. partial results of the planning, can each be stored as data sets in a wireless system, such as via Bluetooth or some other mobile transmission system, or in a wired system, especially in the imaging assistance means, but also in any other storage medium, such as an external computer, etc., or in archiving systems.

The final planning result, hence the image of the planned post-operative condition of the patient, can be shown especially in a navigation view of the display of the imaging assistance means, where instead of the implant models guide lines are displayed, which show the orientation and positioning of the implant.

Also, the final planning result of a possible surgical intervention to be planned and/or partial results of the planning can, for example, be transmitted via Bluetooth or other mobile transmission systems, or in a wired system, including on mobile terminals such as smartphones, tablet computers or notebooks, displayed there, processed and/or stored.

According to the invention, the final planning result of a potential surgical intervention to be planned and/or partial results of the planning can be forwarded via an interface to an apparatus which does not form part of the imaging assistance means. It is possible for data and/or commands to be transmitted to external devices, for example to a further imaging assistance means in an operating theatre.

In this way the final planning result of a potential surgical intervention to be planned and/or partial results of the planning can be shown in the operating theatre.

External devices may be imaging assistance means, display or auxiliary devices, measuring equipment or preferably a mobile or a stationary (operation) navigation system. The navigation system is meant especially for controlling and monitoring a surgical intervention, in particular to determine the local position and/or to determine the coordinates of the intervention and the introduction and the implementation of surgical measures. The planning data may, for example, be transmitted by wireless or wired means via appropriate protocols.

The interface can also be configured such that it is capable of supplying remotely located display devices with information. That may, for example, be a screen located in an operating theatre.

The interface can be configured in such a way that it can do more than just accept data from external sources or supply control data to a display means. If the interface is equipped appropriately, it is possible permanently to load it with data from external sources, to compare them with stored data and to supply them to the surgeon via a display means. Possible data in addition to the image data described above are also geometrical data obtained by external measuring equipment, such as data on the position, orientation or progress of a surgical intervention.

Among other things, it is possible in this context to lay out “electronic fences”, in order to delimit the area of the operation for example, and/or to mark particular regions in the patient's body so as to achieve accurate positioning of an implant in the multidimensional, preferably three-dimensional, planning and to enclose and/or exclude those regions and also to carry out the local coordination of the intervention and/or implant and to transmit the underlying coordinates to a mobile or stationary operation navigation system by wireless or wired means.

It is also possible to translate the data obtained during the pre-operative planning into a 3D model of the implant and to transmit those data via the interface to machines connected to the latter. These may, for example, be cutting machine tools which either make a new implant or rework existing implants individually. It is likewise possible to operate a 3D printer, which either produces an adapted implant or generates a sample that is not suitable as an implant, which can be used for study purposes during the pre-operative planning, depending on the requirements, which is suitable for use as a model for teaching purposes or which can also serve as a model for an implant to be produced by casting.

A further possibility is that the results of the pre-operative planning are transmitted to a navigation system via the interface, and either control data are transmitted directly or the navigation system itself obtains control data from planning or image data received.

It is likewise possible for the imaging assistance means to do more than just supply data via the interface to apparatuses connected up. A retransmission of data from the external apparatuses to the imaging assistance means can likewise be carried out via the interface. This makes it possible, by means of a constant comparison of the target data, planning and actual data, to detect changes that occur during the operation, in order in effect to enable monitoring of the operation performed.

In addition to the automatic collection of data and the automatic comparison, additional visual monitoring of the processes in the body part to be operated on or in the body is possible, and processes can be seen which the surgeon might perhaps not be able to recognise.

If the imaging assistance means or the interface can perform or enable monitoring, it is ensured hereby to transmit via the interface also data or commands to external apparatuses connected up. Especially when a navigation system is used, this gives rise to the possibility of drawing attention to deviations from planned steps and the planning result and in this way of avoiding irregularities in the performance of the operation. The nature of the indications given can depend on the capabilities of the navigation device. Visual and/or acoustic information can be output, for example.

The imaging assistance means can output image data to a monitor in the operating theatre via the interface. In cases in which the surgeon needs to follow up details from the pre-operative planning or current changes, gesture control can be coupled to the imaging assistance means via the interface. This gives rise to the possibility of checking quickly, despite the hygienic conditions prevailing in the operating theatre, whether the operation and its results correspond to the requirements of the planning result.

According to an advantageous aspect of the invention, the planning result and/or partial results of the planning are transmitted to an imaging assistance means in the operating theatre or a navigation system (47). There, the working representation can be activated as navigation view so that the planning result and/or partial results of the planning are shown with guide lines.

This planning result can be correlated with patient images, especially 2D and/or 3D patient images via the interface of the imaging assistance means. In this context one or several X-ray images, CT, MRT or other images generated in an imaging method are taken of the patient in the position on the operating table.

It is also possible to calibrate the image with known methods. With the aid of the interface of the imaging assistance means, a correlation of the coordinates of the plurality of patient images is established so that the patient image of the patient on the operating table corresponds in coordination, orientation and representation to the image of the planning result in the imaging assistance means, especially the navigation view.

Via the interface of the imaging assistance means and the navigation system it is also possible to supply the planning result and/or the parts of the planning result and/or at least the patient image of the patient made on the operating table to an imaging device, for example an X-ray apparatus, an MRT apparatus or a CT apparatus. It is also possible via the interface of the imaging assistance means and the navigation system to supply to the imaging assistance means and the navigation system data and information, as well as to store, display and represent such data and information.

According to the invention, the planning result and/or parts of the planning result and/or patient images showing the patient on the operating table can be transmitted to an image monitoring device by wireless or wired means. The image monitoring device determines virtually according to known methods the location and position of the patient as well as the location, position and progress of individual steps of the planned operation.

Due to the calibration the actual measurements and dimensions of the patient are stored by means of the imaging assistance means and can be transmitted to the navigation system via an interface.

Instruments or implants are likewise stored with the aid of the imaging assistance means for example from the planning according to the method or are supplied with the aid of the imaging assistance means, for example by having access to known information stored or through imaging and calibrated images of the instruments or implants.

The imaging assistance means and the navigation system enable an automatic correlation of the position, dimensioning and location of the patient with the position, alignment and location of implants or instruments.

In addition, the instruments or implants can be connected to at least one location-identifying means which can transmit the relative position of the instruments or implants and changes in position, dimensioning and location of the instruments or apparatuses to the imaging assistance means and the navigation system.

With the aid of the imaging assistance means and the navigation system, all tools, implants and the position of the patient can be shown relative to one another, such as they are actually located in the room.

Especially the guide lines of the planned interventions and the planned implants as well as electronic fences are shown with the aid of the navigation system (47) or the imaging assistance means. Hence, the surgeon is able to adapt in a real-time comparison the individual steps of the operation to the steps of the operation planned before the operation. According to the invention, the surgeon can in particular adapt location, position, angle, depth, positioning and implementation of the steps planned before the operation to the planning result. Hereby the surgeon can avoid that steps of the operation are carried out too deep and/or at an incorrect angle or at an incorrect place.

According to the invention, an apparatus is also provided for performing a surgical intervention in line with the planning result for a surgical intervention obtained in accordance with the invention and/or partial results of the planning, and the apparatus is used to perform a surgical intervention planned in accordance with the invention.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

What is disclosed herein may take physical form in certain parts and arrangement of parts, and will be described in detail in this specification and illustrated in the accompanying drawings which form a part hereof and wherein:

FIG. 1 shows a schematic flow chart,

FIG. 2 shows a screen with two windows,

FIG. 3 shows a window with a sketch illustration and a window with a 3D image,

FIG. 4 shows a spine sketch with a portion of a vertebra and an associated 3D image,

FIG. 5 shows a window with a 3D image and a window with a 2D image,

FIG. 6 shows a screen with four windows, and

FIG. 7 shows two windows with an implant.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices may be shown in block diagram form in order to facilitate describing the claimed subject matter.

In the following, the method of the invention will be described with reference to planning a surgical intervention on a vertebra in the spine of a human being. This, however, is in no way understood in a limiting or exclusive sense, since the method of planning the surgical intervention can also be applied to other parts of the human or animal body.

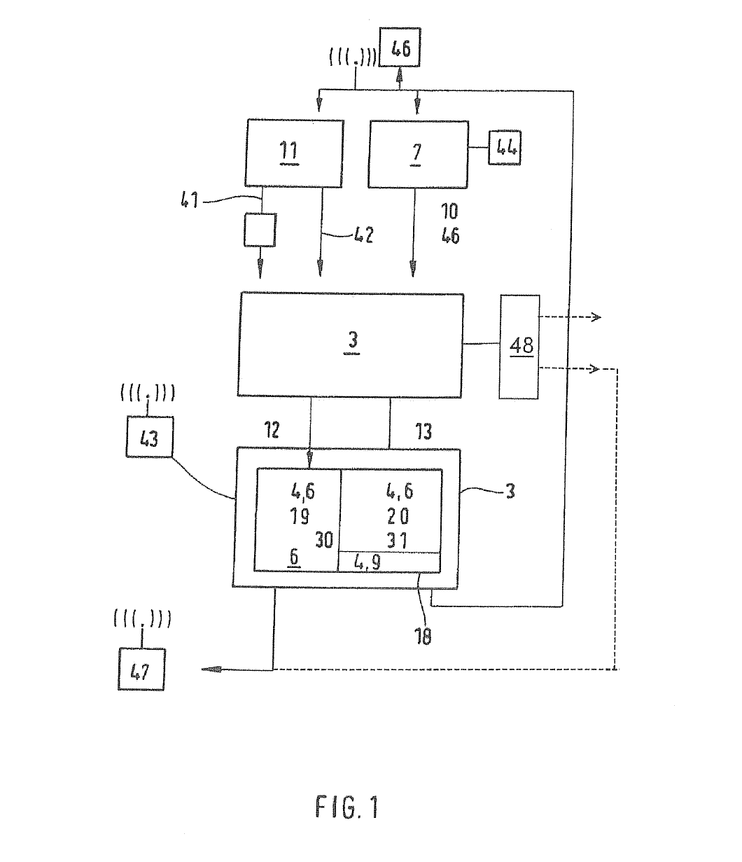

FIG. 1 shows the steps of the planning process of the invention, where the various steps do not necessarily have to be performed in this order and in direct succession.

In a first step, the planner/surgeon selects a number of different multidimensional patient images, especially 2D and/or 3D patient images 41, 42, from an external image source, the storage medium 11 (e.g. a hard disk of an external computer, a USB stick, a CD or DVD or an image archive data program, a PACS system of a hospital, a medical image archive or a similar stationary and/or mobile storage medium), which are supplied to the imaging assistance means 3, e.g. imported into them.

In this context, a number of different multidimensional patient images which are to be supplied to the pre-operative planning can be selected simultaneously.

For the planning, it is in particular possible for several patient images, which may for example have been created at different points in time and/or represent different dimensions, to be imported into the imaging assistance means 3.

Via the external storage medium 11, further reference data 10, such as the height, weight, age and previous illnesses of the patient or data on the medical history so far may be supplied to the imaging assistance means 3 in addition.

In a first intermediate step, the 2D patient images selected are each processed before, while or after being supplied in the imaging assistance means 3, in particular by being scaled so that dimensions of the portion of the patient's body displayed, especially the spine, and/or the scale of the image are supplied with the aid of the imaging assistance means 3.

In the display of a 2D or 3D patient image, the scaling can be performed manually, e.g. by using a reference object, such as a sphere made of non-radiolucent material, or semi-automatically, or automatically, i.e. with computer assistance.

The scaling procedure can be carried out and repeated separately for each individual patient image imported.

Before, during or after the scaling of the individual 2D patient images selected, selected volume-based patient images, i.e. 3D patient images, are supplied to the imaging assistance means in a second intermediate step, i.e. imported into it for example. In the process, in particular information on the scaling of the 3D patient images and/or the format (e.g. CT, MRT, etc.) of the 3D patient images are imported into imaging assistance means 3 from the storage medium 11.

In a third intermediate step, the imaging assistance means 3 may in addition be connected to an external computer 7 in order to take over patient information, such as patient images, dimensioning data, functional data and/or other reference data 10, and/or information on implants 2, such as 2D and 3D models 46 of implants 2, manufacturers' information, article numbers, dimensioning, material, instructions for use etc.

In this context, reference data stored in an external computer 7 or in a PACS system and/or further predefined reference data 10, especially ideal and/or reference models and/or a collection of patient images from different patients concerning parts of the body relevant to the operation, such as the spine, can be imported into the imaging assistance means 3. The external computer 7 may, for example, also be memories based on magnetic, optical or semiconductor structures, such as a hard disk of an external computer, a USB stick, CD or DVD, or an archive data program or a similar stationary and/or mobile storage medium.

In a second step, volume-based patient images, or 3D patient images, can each be segmented with the aid of the imaging assistance means 3, i.e. the 3D patient image concerned is scanned in an analysis that may, for example, be computer-based, automatic, semi-automatic or manual, for bones for instance, especially vertebral bodies.

The body part scanned is identified and provided with the correct medical/anatomical name and/or numbering.

In this segmenting procedure, vertebral bodies and intervertebral disks, for example, are identified and highlighted by a computer-based analysis. In the process, different bone points are compared by means of a comparison with reference data 10 and, having been marked visually with the aid of the imaging assistance means, are highlighted especially on a screen. The comparison can also be performed with computer assistance.

The segmenting procedure can be carried out and repeated separately for each individual 3D patient image imported.

In this way, it can be established on the patient images supplied which skeleton parts, bones, tissues, soft-tissue parts etc., are present in the patient concerned, how they are arranged spatially, what the medical terms are for the individual body parts and/or, where applicable, whether and what native or pathological changes are present compared to the reference data 10. It is also possible to visualise the position, name etc. of skeleton parts, bones, tissues, soft-tissue parts etc. in the patient image concerned by displaying a sketch in a further window.

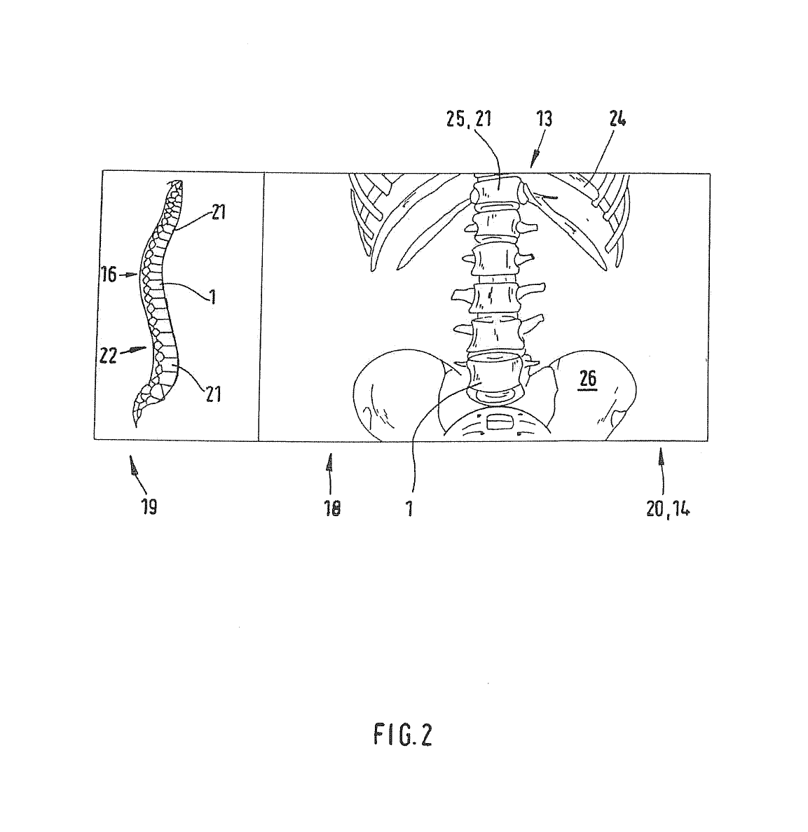

This is illustrated by way of example in FIG. 2.

FIG. 2 shows a screen 18 with two windows 19 and 20. One window 19 shows a sketch 16 of a spine with vertebrae 21. In the lower region of the vertebrae 21, a portion 22 of the vertebrae 21 is shown. The portion 22 of the vertebrae 21 is marked by a cursor (not shown).

In the second window 20, a segmented vertebra 14 of the body 1 (spine) is shown, in which only the vertebrae 21 marked in the window 19 are displayed.

The window 20 shows the body 1 with its vertebrae 21 in a 3D image 13. In the upper portion of the window 20, the thorax 24 is illustrated rudimentarily. The thorax 24 with its vertebrae 21 is connected to the pelvis 26 via the spine 25.

Segmenting, which may, for example, be computer-based, automatic, semi-automatic and/or manual, enables the planner/surgeon in a first intermediate step to use the imaging assistance means 3 in order to check the results obtained in computer-based segmenting, such as the positioning of bones, especially vertebrae, tissues, soft-tissue parts etc., their spatial arrangement in the patient images and/or their medical names.

The planner/surgeon is also enabled to correct the segmenting, i.e. if a bone, especially vertebral body, was not identified with computer assistance by the contours shown in the 3D patient image, and to rework the segmenting and naming manually in a second intermediate step with the aid of the cursor 23 for instance.

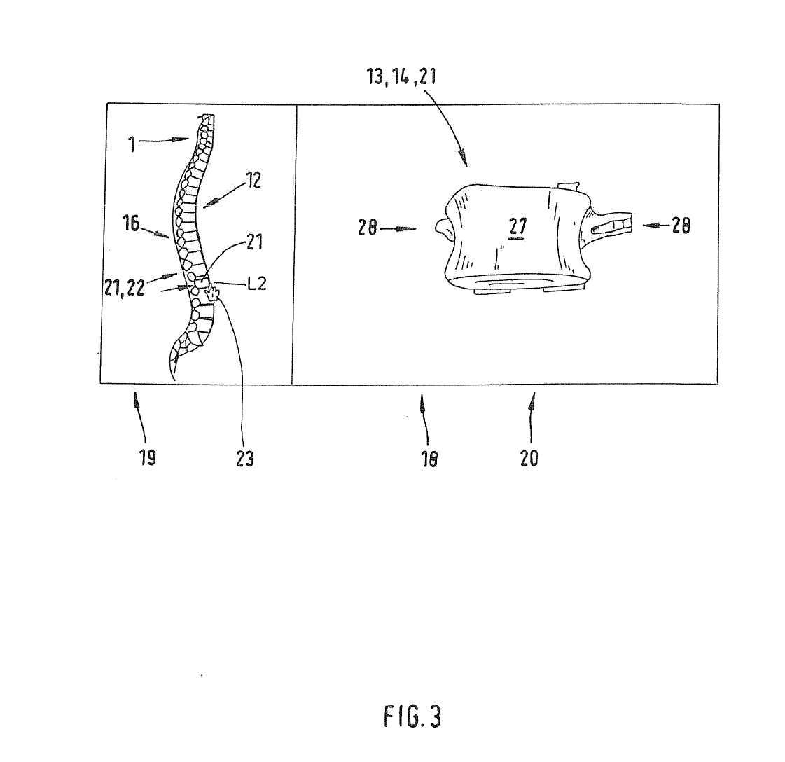

On this subject, FIG. 3 likewise shows a screen 18 with two windows 19 and 20, one window 19 of which is a 2D image 12, and the adjacent window 20 of the screen 18 shows a segmented vertebra 21, 14. The segmented vertebra here is shown as a 3D image 13.

In the sketch 16 of the spine, the portion 22 of the vertebrae 21 of the body 1 is marked. The cursor 23 marks a particular vertebra 21. The marked vertebra 21 is now highlighted and marked in a different colour from the adjacent portions 22 of the vertebra 21.

In the window 20 of the screen 18, the vertebra 21 marked in the sketch 16 of the spine is shown as a 3D image 13. The vertebra 21 comprises a vertebral body 27 and transverse processes 28.

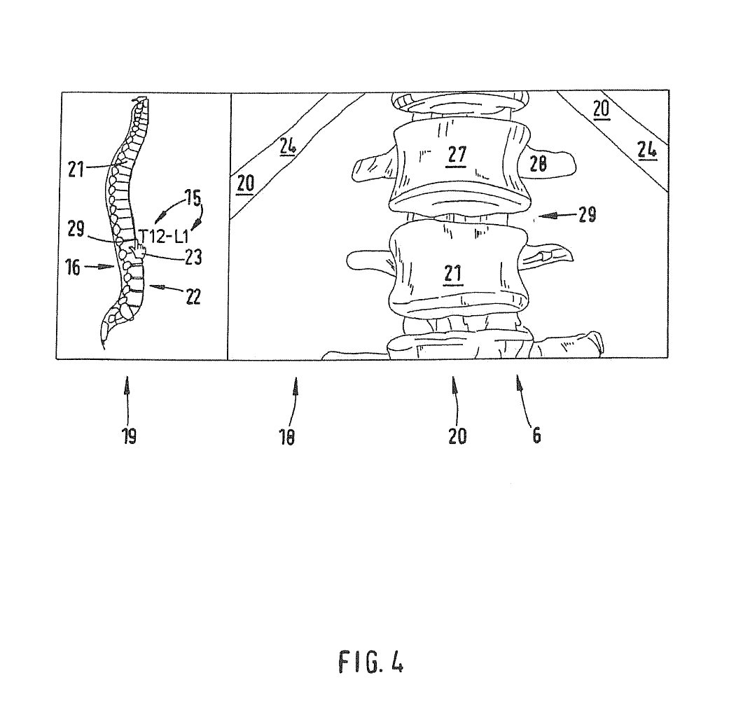

The examination of the segmenting can be performed by marking regions of the image of bones, tissues and soft-tissue parts, as illustrated in FIG. 4.

By means of the comparison with reference data 10, the planner/surgeon can highlight bones, especially vertebrae, and tissues etc., in colour with the aid of the cursor 23.

FIG. 4 shows a window 19 and a window 20 of the screen 18 comparable to the representation from FIGS. 2 and 3.

In the left-hand window 19, the sketch 16 of the spine is shown, which includes vertebrae 21. A portion 22 of the vertebrae 21 is marked and, comparable to FIG. 3, is provided with a name 15 “L1”. The cursor 23 is located in the position of the intervertebral disk 29 which has been provided with the name “T12”.

The second window 20 is configured as a working representation 6 and shows the vertebrae 21 with vertebral bodies 27 and transverse processes 28.

Corresponding to the mark made with the aid of the cursor 23 in the sketch 16 of the spine, the intervertebral disk 29 is highlighted. To the left and right of the vertebral body 21, ribs 39 of the thorax 24 are displayed in the window 20.

In a third step, it becomes possible to correlate bones, especially vertebrae, tissues, soft-tissue parts etc., displayed in the 2D or 3D patient images with the 2D or 3D patient images respectively in the correct location by means of a fixed coordinate reference between the 2D image data and the 3D image data. This results in a reference in the coordinate system concerned, so that simultaneous representations of the same coordinate in the 2D and 3D patient images can be displayed in different windows.

The coordinate reference can be created with computer assistance or manually.

When the coordinate reference is created manually, the cursor 23 is used in order, for example, to visually enhance contour lines of bones, especially vertebrae, such as superior and inferior endplates of vertebral bodies in the spine, in the 2D or 3D patient images with the aid of the imaging assistance means 3 and to mark them in colour, for example. An example of outlining superior and inferior endplates of vertebral bodies in the spine can be seen in FIG. 4. There, with the aid of the imaging assistance means 3, the cursor 23 has been used to draw two demarcation lines on each bone body, especially the vertebral bodies, and those lines have the name of the vertebral body concerned assigned to them in order to create the coordinate reference.

In a fourth step, the planner/surgeon 43 selects at least one 2D and/or 3D image 12, 13 for display in at least one window of the screen 18 of the imaging assistance means 3 as a working representation 6, which is used to continue the pre-operative planning.

The selection of at least one of the images 4 in a window 19, 20, 30, 31 as a working representation 6 by the planner/surgeon 43 makes it possible to perform further planning steps in that image, especially processing steps and/or simulations.

The 2D and/or 3D images 12, 13 selected can each be displayed as images in different windows 19, 20, 30, 31 on a screen 18 of the imaging assistance means 3.

The planner/surgeon 43 preferably selects a 2D and a 3D patient image, between which a spatial coordinate reference has been created. That makes it possible, for example, to display different load situations, especially of vertebrae and joints, different postures of the patient, different recording angles and/or surfaces etc. simultaneously and thus to include them in comprehensive pre-operative planning.

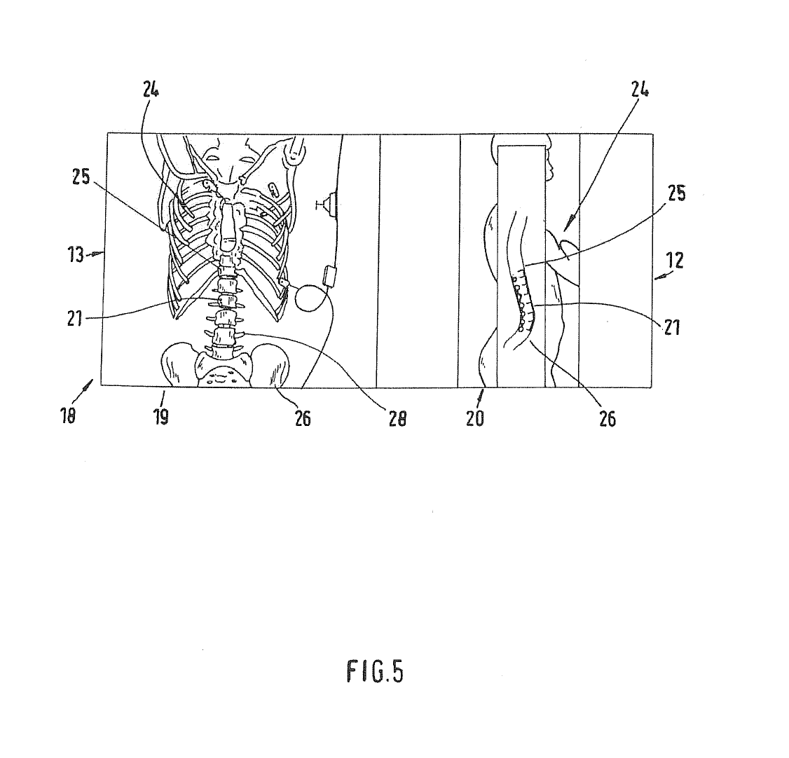

An example of an image can be found in FIG. 5. FIG. 5 shows a window 19 and a window 20 of the screen 18 comparable to the representation from FIG. 2 to FIG. 4. In the left-hand window 19, a 3D patient image 13 of the spine 25 is shown, which includes vertebrae 21. The second window 20 shows a 2D patient image of the spine 25 with the vertebrae 21.

Thanks to the coordinate reference, a measurement, marking, or simulation of the introduction of an implant in a 2D or 3D patient image can be transferred with computer assistance to another 2D or 3D patient image with the aid of the cursor 23, i.e. in at least one image in at least one window 19, 20, 30, 31, marking etc. is carried out, and that is transferred to another image and/or window and displayed based on coordinates.

This transfer can preferably be made during the planning of operations on the spine in the sagittal and axial balance in order to determine intervertebral disk angles, such as in the case of lordosis and kyphosis, as illustrated in FIG. 5.

Pre-operative planning is, however, also possible with a simultaneous display of different multidimensional patient images in different windows 19, 20, 30, 31 without any coordinate reference.

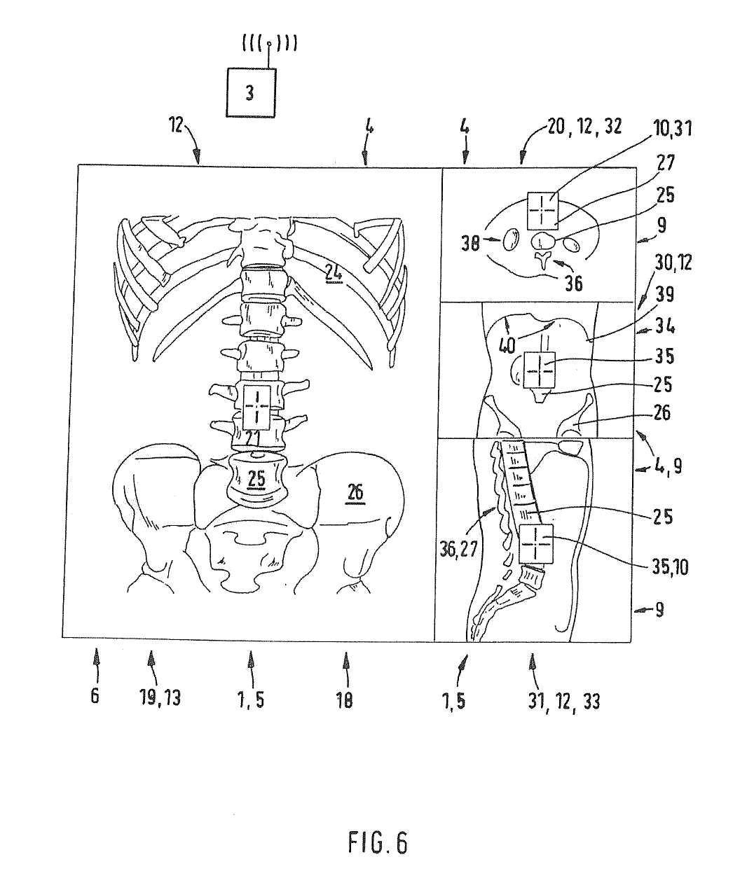

On this subject, FIG. 6 shows a screen 18 with a window 19 and further windows 20, 30 and 31.

The window 19 shows a three-dimensional image 13 of the body 1 with the thorax 24, spine 25 with vertebrae 21, and the pelvis 26. The image in the window 19 of the screen 18 is the working representation 6.

The windows 20, 30 and 31 are each three-dimensional images 13, which respectively show an axial section 32, a sagittal section 33 and a coronal section 34 through the spine 25.

With the aid of the imaging assistance means 3, a navigation cross 35 can be displayed in the images 4 in the windows 19, 20, 30, 31. The navigation cross 35 comprises three axes, one axis of which runs from top to bottom, the second axis runs from left to right, and a third axis, which runs from front to back through the patient's body 1.

The window 20 shows the original 3D image 13, from which the other images 4, 9 in the other windows 19, 30, 31 can be calculated.

The image 4, 9 in the window 20 shows the axial image of the spine 25 from top to bottom. On the rear of the spine 25, a spinous process 36 of a vertebral body 27 is shown. Opposite the spinous process 36 in front of the spine 25 the patient's aorta 37 is shown. To the left and right of the spine 25, the kidneys 38 are displayed in the window 20.

The image 4, 9 in the window 30 shows the patient in the region of the navigation cross 35, seen from the front. To the left and right beneath the navigation cross 35, the bones of the pelvis 26 are displayed. In the centre of the image 4, 9 in the window 30, the spine 25 runs from top to bottom.

Approximately parallel to the axis of the spine on a line of body tissue, the attachment points of the ribs 39 can be seen. The lobes of the lungs 40 are illustrated between the ribs 39.

A sagittal section 33 through the spine 25 is shown in the window 31 of the screen 18. As an orientation aid for the surgeon, the navigation cross 35 is also illustrated in the window 31. Approximately parallel to the axis of the navigation cross 35 running from top to bottom is the spine 25. The spinous processes 36 of the vertebral body 27 can be seen on the rear of the spine 25.

In a fifth step of the method, the planner/surgeon 43 uses the imaging assistance means to carry out further planning steps in at least one image 6, especially to simulate a surgical intervention to be planned.

All the steps of a surgical intervention to be planned, especially to introduce an implant, can be simulated in each of the images 4, 6, windows 19, 20, 30, 31 and/or the 3D or 2D image 12, 13, such as a complete replacement of an intervertebral disk, the insertion of a cage and/or stent, the cementing on of bones, especially of vertebrae, the treatment of cracks in an intervertebral disk, treatments of arthritic situations, the treatment of bone fractures, the insertion of screws, especially pedicle screws, the insertion of plates, bars, etc., the grinding of bone, ulti-mately therefore the steps of the surgical method or procedure.

The simulation is performed by selecting, mapping and moving a 2D and 3D model 46 of an implant 2 with the aid of the cursor 23 in at least one working representation 6.

In order to simulate the intervention, the planner/surgeon 43 can select 2D and 3D models 46 of implants 2 supplied with the aid of the imaging assistance means, such as cages (implant cages), components of artificial joints, screws, plates, distraction-compression apparatuses, implant components, alignment guides, disposable instruments etc., and also information 46 on the respective implants and/or fixing means for implants 2, 46, such as manufacturers' information, article numbers, sizes, properties, illustrative material, dimensioning, size, length, diameter, shape, material, instructions for use etc.

The 2D and 3D models 46 of implants 2, and information on the respective implants and/or fixing means for implants may be stored in a database, preferably in the computer 7 or some other external data memory 44, such as a hard disk of an external computer, a USB stick, CD, DVD, an archive data program, a cloud system or a similar stationary and/or mobile storage medium.

The 2D and 3D models 46 of implants 2, information on the respective implants and/or fixing means for implants can be displayed and moved in the images 4, 6, the windows 19, 20, 30, 31 and/or the 3D or 2D images 12, 13.

With the aid of the imaging assistance means, it is in particular possible to display dimensioning, such as the length, depth, circumference, height and width and/or density of, for example, a bone and/or part of a skeleton, a tissue, soft-tissue part or organ, in the form of reference points, by means of which reference points on implant models 46 can be compared and in this way automatically or manually to select and propose matching implants, such as cages (implant cages), components, artificial joints, screws, plates, distraction-compression apparatuses, implant components, alignment guides, disposable instruments, securing means, acetabula and shanks for hip implants etc.

In a preferably automatic selection process for planning a surgical intervention, a comparison is made between known and/or identified reference points between implant information and the patient images and is used to determine and propose at least one most suitable implant 2, which is automatically superimposed in one of the images, e.g. in the 3D patient image 13, with the aid of the imaging assistance means.

The individual steps of the surgical intervention can be simulated and/or illustrated in this way.

Among other things, it is possible to select an implant, the position of the implant in the patient's body and the spatial coordinates for the introduction of the implant for the intervention, and to decide on the beginning of the intervention and also the aids for fixing the implant and their spatial arrangement.

It is also possible to define “electronic fences”, in order to delimit the area of the operation for example, and/or to mark particular regions in the patient's body so as to achieve accurate positioning of an implant in the multidimensional, preferably three-dimensional planning and to transmit information on the electronic fences via an interface to external devices, for example a navigation system or an imaging assistance means in an operating theatre in a wireless or wired system.

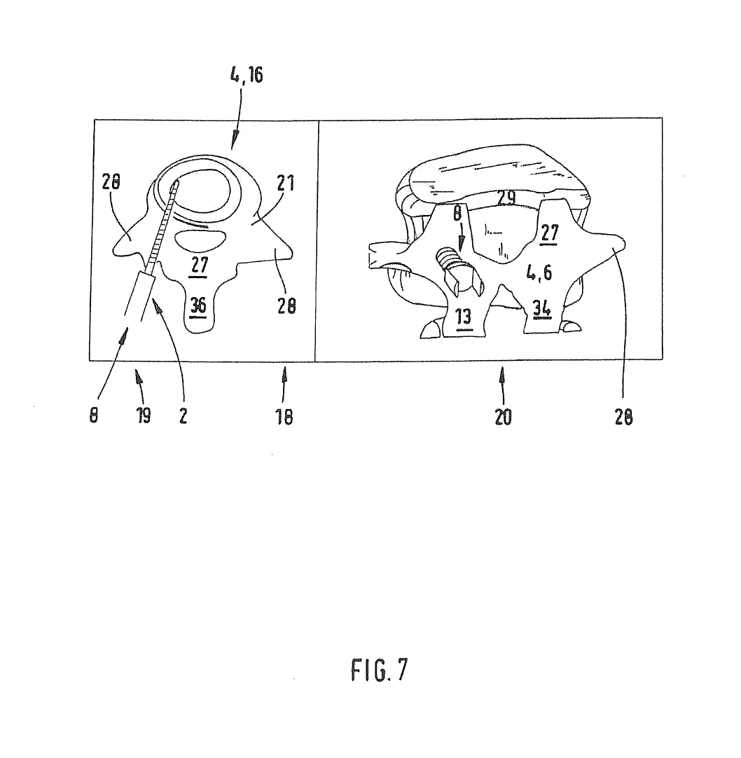

In this connection, FIG. 7 shows the simultaneous display of an implant model, a patient image and a working representation in the simulation of how a screw can be inserted into a vertebra.

FIG. 7 shows the screen 18 with windows 19 and 20. In this case, the image 4 is a vertebral body sketch. The image 4, 6 is displayed in the window 20 as a three-dimensional image 13.

The spine sketch 16 shows the vertebra 21 with a vertebral body 27 and the transverse processes 28 and a spinous process 36. In the region of the left-hand transverse process 28, an implant model 2, 46 in the form of a screw is illustrated.

The positioning 8 of the implant model 2 is identical, with regard to the orientation, the set angle relative to the vertebral body 27 and relative to the configuration of the screw 2, to the positioning 8 and the configuration of the screw in the sketch of the window 19.

In the vertebral body 27, a coronal section plane 34 has been drawn in, in which the transverse processes 28 and an intervertebral disk 29 can be seen.

In a further optional step, the planning steps for a possible surgical intervention to be planned and/or the final planning result of a surgical intervention can each be stored as data sets in a wireless system, such as via Bluetooth or some other mobile transmission system, or in a wired system, especially in the external computer 7 and/or in archiving systems such as the storage medium 11.

In a further optional embodiment of the method, all the data can also be transmitted in a wireless or wired system to mobile terminals 45 and displayed there.

In a further advantageous embodiment of the method of the invention, the planning data obtained, especially the planning result and/or partial results of the planning, can be transmitted via an interface to a mobile or stationary operation navigation system 47 or an imaging assistance means. The transmission takes place in a wireless or wired system for example in an operating theatre, in order to determine the local position and/or determine the coordinates of the intervention and to initiate surgical steps in accordance with the planning data.

In the process, it is in particular possible to export the spatial arrangement, “electronic fences”, and the underlying coordinates to a mobile or stationary operation navigation system 47 in a wireless or wired system.

A navigation system of this kind is an apparatus for performing a surgical intervention which has been planned according to the method of the invention.

A navigation system of this kind may in particular be used for performing a surgical intervention which has been planned according to the method of the invention.

Due to the calibration the actual measurements and dimensions of the patient are stored by means of the imaging assistance means and can be transmitted to the navigation system via an interface.

Instruments or implants are likewise stored with the aid of the imaging assistance means for example from the planning according to the method or are supplied with the aid of the imaging assistance means, for example by having access to known information stored or through imaging and calibrated images of the instruments or implants.

The imaging assistance means and the navigation system enable an automatic correlation of the position, dimensioning and location of the patient with the position, alignment and location of implants or instruments.

In addition, the instruments or implants can be connected to at least one location-identifying means which can transmit the relative position of the instruments or implants and changes in position, dimensioning and location of the instruments or apparatuses to the imaging assistance means and the navigation system. Location-identifying means can be, for example, electromagnetic location-identifying systems, optical location-identifying systems, acoustic location-identifying systems. The location-identifying means can also send information, signals or radiation in a wired or wireless system especially to the navigation system or the imaging assistance means or to the navigation system and the imaging assistance means, or receive or send and receive such information, signals or radiation.

With the aid of the imaging assistance means and the navigation system, all tools, implants and the position of the patient can be shown relative to one another, such as they are actually located in the room.

With the aid of the imaging assistance means and the navigation system the location, dimensioning and position of various instruments and implants, for example catheters, spine implants, samples etc., can be identified and displayed. With the aid of the imaging assistance means and/or the navigation system the image can be displayed two-dimensionally as well as three-dimensionally. In particular, the location, dimensioning and position of these instruments and implants relative to the location, dimensioning and position of the body of the patient can be shown in a two-dimensional or three-dimensional manner.

Position, location and dimensioning of the implants and instruments can be transmitted in a wired or wireless system to the imaging assistance means or the navigation system or the imaging assistance means and the navigation system continuously or at time intervals.

In particular, the imaging assistance means can store, display and retrieve different coordinates and display them relative to other coordinates.

Thus, with the aid of the method according to the patent changes in the position, location and alignment of the instruments, and relative to the body of the patient, can be displayed and monitored. The surgeon can monitor with the aid of the imaging assistance means and the navigation system whether the steps of the operation that are carried out correspond to the planning result and/or partial results of the planning, and make corrections if necessary.

The imaging assistance means 3 of the invention above all includes an interface 48 via which online data can be transmitted during the surgical intervention to display, auxiliary, measuring and/or image recording equipment.

In a preferred embodiment, the imaging assistance means 3 of the invention can also retrieve data from the above-mentioned equipment via the interface 48.

While operations are being carried out with imaging methods, it is common for pictures to be taken and displayed in the operating theatre in the conventional way. The imaging assistance means 3 of the invention now has the possibility via the interface 48 of establishing connections with those imaging or image-displaying devices and taking over data available there.

The imaging assistance means 3 can compare the data retrieved with the planning data and identify any differences that have arisen.

A preferred embodiment of the imaging assistance means 3 is able to process data received via the interface 48 in real time, to derive information from them and to transmit it back to the equipment connected up, likewise in real time. In this way, deviations in the course of the operation from the operation planning can be identified and indicated.

Deviations of this kind may, for example, be unintended changes in the position of the patient operated on, errors in the axes of the bones or bone parts to be operated on or the fact that the boundaries with out-of-bounds areas, also known as digital gates, are being approached or crossed.

After an operation has been completed or also after the introduction of one or more implants and before the closure of the operation wound, the imaging assistance means 3 can likewise carry out a final measurement via the interface 48 with peripherally arranged equipment in order to determine the success of the operation.

It is also possible, parallel to the surgeon, to employ a second planner on the imaging assistance means 3, who observes the operating procedure, compares the working steps or working results with the planning data and intervenes if necessary.

More advanced possibilities can be opened up via the interface 48 of the imaging assistance means 3.

Via the interface 48, a remote display unit can be connected up, which shows not only pictures from the operating theatre during an operation, but also the planning data for the operation and/or results of measurements while the operation is being performed. This can be used for teaching purposes for example.

During the planning process and before beginning an operation, the planner/surgeon 43 can transmit planning data to a navigation apparatus 47 used in the operating theatre and, in a further embodiment, can also program it.

In the course of or after completion of a planning process, the planner/surgeon 43 can decide on the form, dimensions and any special conditions for a necessary implant 2. Via the interface 48 of the imaging assistance means, he can output control data generated by the imaging assistance means 3 to a 3D printer, which uses the control data to generate a model of the implant 2 required.

The models of the implants 2 produced in this way can be used by the planner/surgeon 43 as a physical embodiment enabling him to examine the implant in the original, make alterations to it and then continue the planning for the operation.

It is likewise possible to use the models generated by a 3D printer for demonstration and teaching purposes.

If suitable materials for 3D printing are available, it is also possible to generate implants 2 in this way.

A further possible use of the imaging assistance means 3 can be that, during the planning of an operation, a suitable implant 2 is selected, but one that requires reworking. In such a case, control data can be transmitted via the interface 48 of the imaging assistance means 3 to a cutting machine, which reworks the implant 2 in accordance with those control data.

A further possible use of the imaging assistance means 3 is that input devices can be connected to the imaging assistance means 3 via the interface 48. This makes it possible for commands to be transmitted to the imaging assistance means 3 from the operating theatre during an operation, which processes the commands and, for example, transmits information to peripheral equipment connected up.

All known possibilities are conceivable as input devices. These are in particular a computer available in the operating theatre with input possibilities, a tablet computer, notebook, smartphone or equipment allowing control by gesture.

It is not necessary for the planner/surgeon 43 to operate that equipment himself. Nevertheless, if he uses gesture control, he may for instance cause the imaging assistance means 3 during the operation to project particular 2 or 3-dimensional images of the operation area onto a display device and, for example, to rotate their position, change their size, highlight them in colour or the like. In combination with the possibilities described above, the planner/surgeon 43 can at any time get a picture of the actual circumstances in the area operated on.

Depending on the requirements of the peripheral equipment to be connected to it, the interface 48 of the imaging assistance means 3 can operate by wireless or wired means. A combination of the two possibilities is likewise possible.

The imaging assistance means 3 can generate due to the planning results and/or the partial results of the planning control data and transmit them in a wireless or wired system so as to drive peripheral apparatuses directly.

It is also possible to generate the control data due to the planning results and/or partial results of the planning according to the invention in accordance with particular protocol requirements which apply to peripheral equipment to be connected.

The method of the invention for planning, supporting, monitoring and/or subsequently checking a surgical intervention and the apparatus of the invention used for that purpose involve advantageous possibilities for arriving at an operating procedure that can be completely planned, monitored and controlled, in which it is additionally possible to make use of known image data and third-party data stocks and to employ comparison and selection processes both with regard to the technical equipment required, the circumstances of the patient and the requirements and properties of the implant in order to decide on all the steps required.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Of course, those skilled in the art will recognize many modifications may be made to this configuration without departing from the scope or spirit of the claimed subject matter.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure.

In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

What is claimed is:

1. A method of planning, preparing, supporting, monitoring and/or subsequently checking a surgical intervention in the human or animal body (1), in particular for introducing at least one implant (2), in which, with the aid of an imaging assistance means (3), at least two multidimensional images (4, 12, 13) of at least one portion (5) of the body (1) are displayed, at least two images (4) of which differ with respect to the number of dimensions of the image (4), wherein the multidimensional images (12, 13) are identified and processed before, while or after they are displayed with the aid of the imaging assistance means (3) and wherein information and/or data on implants (2, 46) and/or 2D and/or 3D images (12, 13) of implants is/are supplied to the imaging assistance means and wherein at least one patient image (12, 13) is identified and processed before, while or after it is displayed with the aid of an imaging assistance means (3) in a working representation and wherein the at least one multidimensional image can be compared with a reference model (10) and wherein the 2D and 3D patient images (12, 13) are correlated with one another and wherein the imaging assistance means (3) uses an interface (48) to transmit data online during the surgical intervention to display, auxiliary, measuring and image recording equipment, and wherein for planning, supporting, monitoring and/or subsequently checking a surgical intervention the imaging assistance means (3) is supplied with reference data, target values, standard values and/or general non-patient-specific data and/or information on implants which, before being supplied, is/are compared with standard values.

2. A method of planning, preparing, supporting, monitoring and/or subsequently checking a surgical intervention claimed in claim 1, in which wherein with the aid of the imaging assistance means before beginning the operation at least one image of the patient is taken in the positioning which the patient adopts during the operation, and wherein information on the planning of the operation is passed to an imaging assistance means in the operating theatre via an interface, and wherein the individual steps of the operation are compared with the steps of the operation planned before the operation with the aid of the imaging assistance means.

3. The method as claimed in claim 1, in which at least one implant model (2, 46) can be positioned in one or more images (4, 9) of the portion of the body (5) with the aid of the imaging assistance means (3), and can be processed with respect to that portion of the body.

4. The method as claimed in claim 1, in which the positioning (8) and/or processing of the at least one implant model (2, 46) with respect to the portion of the body (5) is transmitted to all the images or to at least one image (4, 9) and displayed in it/them (4, 9) with the aid of the imaging assistance means (3).

5. The method as claimed in claim 1, in which the planning steps and/or planning result are stored in the imaging assistance means (3) and/or in some other memory (7, 11, 44).

6. The method as claimed in claim 1, in which control data are output to a 3D printer by the imaging assistance means (3) via the interface (48) based on the results of the planning.

7. The method as claimed in claim 1, in which the results of the planning and/or results of a capture during the operation and/or results of a subsequent check are transmitted to a display unit remote from the imaging assistance means (3) via the interface (48) and the control of the picture displayed can be taken over interactively by an operator.

8. The method as claimed in claim 1, in which it is determinable by means of manual inputs what planning information is passed to a navigation system (47) via the interface (48).

9. The method as claimed in claim 1, in which reference points on the implant are determinable, the reference points are passed to a navigation system (47) via the interface (48) and are compared with the default values by the navigation system (47).

10. The method as claimed in claim 1, in which a navigation system (47) outputs visual and acoustic signals.

11. The method as claimed in claim 1, in which out-of-bounds areas are calculated during the planning of an operation, integrated into the planning and checked during the operation and/or checked after the operation.

12. The method as claimed in claim 1, in which data to demarcate the region of the operation and/or to mark particular regions in the patient's body (1) and/or to position an implant accurately can be input and/or retrieved.

13. The method as claimed in claim 1, which has a measuring and/or image recording device associated with it which supplies data to the imaging assistance means (3) online via the interface (48).

14. An apparatus for carrying out a method of planning, preparing, supporting, monitoring and/or subsequently checking a surgical intervention as claimed in claim 1, which consists of an imaging assistance means (3) with software adapted to said imaging assistance means (3) and an interface (48) for importing data to display, auxiliary, measuring and/or image recording equipment, which has a spatially remote display device associated with it via the interface (48), which has an auxiliary device associated with it which receives control data from the imaging assistance means (3) and which has a measuring and/or image recording device associated with it which supplies data to the imaging assistance means (3) online via the interface (48).

15. Use of an apparatus as claimed in claim 14 for planning, preparing, supporting, monitoring and/or subsequently checking a surgical intervention in the human or animal body.
